(19) United States
(12) Patent Application Publication
(10) Pub. No.: US 2014/0316336 A1
(43) Pub. Date: Oct. 23, 2014

(54) AUTO-DISABLE SAFETY SYRINGE

(71) Applicant: Amer W. Hawasheen, Industrial City of Abu Dhabi (AE)

(72) Inventor: Amer W. Hawasheen, Industrial City of Abu Dhabi (AE)

(73) Assignee: ABU DHABI NATIONAL INDUSTRIAL PROJECTS CO., Industrial City of Abu Dhabi (AE)

(21) Appl. No.: 13/978,643

(22) PCT Filed: Jan. 11, 2011
(86) PCT No.: PCT/IB11/00025

§ 371 Date: Jan. 14, 2014

(51) Int. Cl. A61M 5/32; A61M 5/50

(52) U.S. Cl. CPC A61M 5/322; A61M 5/502; A61M 5/504; A61M 5/5066; A61M 2005/5026; A61M 2005/3128
USPC 604/110

(57) ABSTRACT

A single use hyperdermic syringe (20) having a one-way valve (56, 62) to prevent refilling and a retractable needle (30). A needle connector (26) and a needle hub (28) with needle (30) form an assembly (40) with a cavity (52) for the passage of liquid. An elastically-deformable sealing ring (56) and a movable sealing member (62) in the cavity are configured to form a seal, after a single use of the syringe, against a flow of liquid into the syringe barrel (22). Locking pins (76) on the piston engage the needle connector when the barrel is emptied, whereby the needle is retractable into the barrel by moving the plunger (24) in the proximal direction.

### FIELD OF THE INVENTION

The invention pertains to single use disposable syringes having a retractable needle.

### BACKGROUND OF THE INVENTION

Conventional disposable hypodermic syringes present two distinct health hazards. One is the accidental scratching or puncturing by the needle of the skin of the person administering an injection after with- drawal from the patient or during disposal. The other is the intentional re-use of the syringe, even though intended for a single use, such as re- use by drug addicts. Both hazards can result in the spread of infectious diseases where the syringe is contaminated as a result of use.

Various designs of syringes have been made to address one or the other of these concerns. Mechanisms have been designed for the retraction of the needle into the syringe barrel after use, so that the barrel acts as a protective sheath. Examples in the patent literature include Lake, WO 95/03845 A1; Jenson, U.S. Pat. No. 5,380,285; and Smith et al., US 2004/0087907 A1. Mechanisms which disable the syringe by preventing refilling after a single injection have also been designed, for example Meyer et al., U.S. Pat. No. 5,613,951; Butler et al., U.S. Pat. No. 4,941,879; and Richardson et al., WO 92/04064 A1. Goossens et al., WO 2005/023344 A1 discloses a design in which a closure element blocks the fluid passageway, creating a vacuum in the syringe barrel when the plunger is retracted, withdrawing the needle into the barrel.

The present invention is directed to improvements in the art of single use safety syringes to reduce the foregoing hazards.

### SUMMARY OF THE INVENTION

The invention provides a single use syringe in which the syringe may not be refilled after one use and in which the needle may be withdrawn into the syringe barrel. The syringe has a barrel and a plunger, a needle connector at the distal end of the barrel having a portion within the barrel bore and a portion extending from an opening in the distal end of the barrel, the needle connector being in sealing, releasable engagement with the barrel wall, and a needle hub affixed to the needle connector and having a hypodermic needle fixed thereto. The needle connector and the needle hub form an assembly, the assem- bly having a cavity therein. The needle and the barrel bore are in fluid communication through the cavity. An elastically-deformable sealing ring having an opening and a movable sealing member are positioned in the cavity. The sealing member is movable from a first position in which its head is on the proximal side of the ring to a second position in which the head is on the distal side of the ring; the ring being config- ured to stop movement of the sealing member from the second position to the first position and to form a seal with the sealing member, when the sealing member is in the second position, against a flow of fluid in the proximal direction. This mechanism provides a one-way valve in the syringe. There are locking means for locking together the plunger and the needle connector such that the needle is retractable into the barrel bore, when the plunger and the needle connector are locked together, by movement of the plunger in the proximal direction.

The one-way valve operates after some volume of fluid is ex- pelled during an injection but it does not require that the plunger be in a fully inserted position within the barrel, i.e. that the barrel be emptied. It thus prevents misuse by refilling even where the barrel has been only partly emptied. At the same time, the syringe provides a mechanism for mechanically locking the plunger to the needle assembly, providing for secure retraction of the needle into the barrel after use.

In the description of the syringe herein, “distal” indicates the needle end of the syringe (i.e. the left side in FIG. 1) and “proximal” indicates the opposite, plunger end (i.e. the right side in FIG. 1).

Further aspects of the invention and features of specific embodiments are described below.

### BRIEF DESCRIPTION OF THE DRAWINGS

In drawings in which corresponding and like parts are identified by the same reference characters:

FIG. 1 is a side elevation view, partly cutaway, of a syringe in accordance with a first embodiment of the invention.

FIG. 2 is a longitudinal cross-sectional view of the distal portion of the syringe of FIG. 1, shown ready for injection.

FIG. 3 is a cross-sectional view on the line 3-3 of FIG. 2.

FIG. 4 is a longitudinal cross-sectional view of a portion of the distal portion of the syringe, shown after the injection has commenced, with the movable sealing member in the position which prevents refilling.

FIG. 5 is a longitudinal cross-sectional view of the syringe of FIG. 1, shown at the end of an injection stroke, with the plunger and needle connector locked together.

FIG. 6 is a longitudinal cross-sectional view of the syringe of FIG. 1, shown with the needle withdrawn into the syringe barrel after use.

FIG. 6A is a detailed view of a portion of FIG. 6.

FIG. 7 is a longitudinal cross-sectional view of the distal portion of a syringe in accordance with a second embodiment of the invention, shown after actuation of the movable sealing member and before the locking of the plunger to the needle connector.

FIG. 8 is a view similar to FIG. 7, shown after locking of the plunger to the needle connector.

FIG. 9 is an isometric view of the distal portion of the plunger of the syringe of FIG. 7.

FIG. 10 is a longitudinal cross-sectional view of the distal portion of a syringe in accordance with a third embodiment of the invention, shown after actuation of the movable sealing member and with the plunger locked to the needle connector.

FIG. 11 is an isometric view of the distal portion of the plunger of the syringe of FIG. 10.

FIG. 12 is an isometric view of the needle connector of the syringe of FIG. 10.

### DETAILED DESCRIPTION

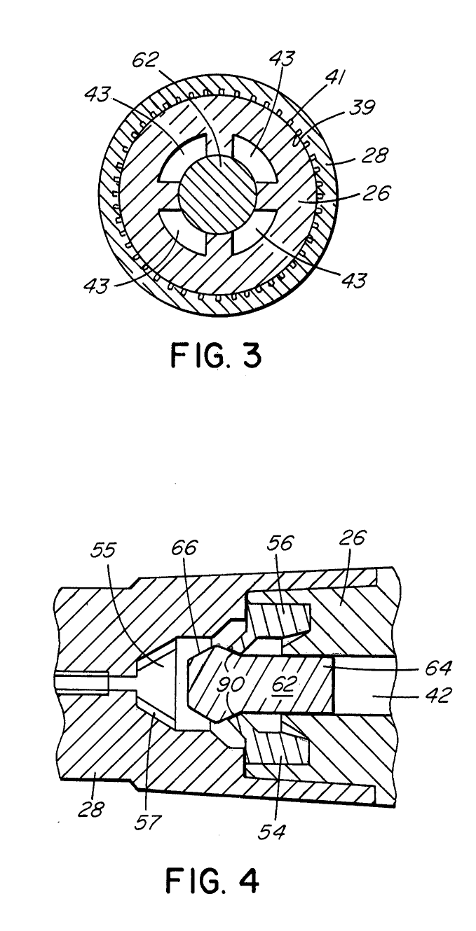

Referring first to FIGS. 1 to 6, which show a first embodiment of the safety syringe, the single use hypodermic syringe 20 includes a barrel 22, a plunger 24, a needle connector 26 and a needle hub 28 with a hypodermic needle 30 affixed to it. The barrel 22 has a barrel wall 23 defining a bore 25. The barrel has a radially outward outwardly-extending finger flange 27 at its proximal end. The plunger 24 includes a plunger shaft or rod 29, a piston 31 at its distal end and a thumb rest 35 at its proximal end. The plunger shaft is cross-vaned in structure. An O-ring 68 on the piston 31 forms a fluid seal against the inner surface of the barrel wall 23.

The needle connector 26 is located at the distal end of the barrel 22 and has a first portion 32 lying within the bore and a second portion 33 which extends out of the opening 34 in the distal end of the barrel. The radially outer surface 36 of the first portion 33 of the needle connector sealingly engages against the inner surface 38 of the barrel wall, such that fluid in the barrel bore 25 cannot escape between them. The radially outer surface 36 of the needle connector and the inner surface 38 of the barrel wall are, at their point of engagement, con- toured to create a releasable snap-fit of the needle connector within the barrel. The snap-fit mechanism is such that the needle connector can be withdrawn into the barrel for purposes of sheathing the needle, as described below, but it is sufficiently strong to prevent movement of the needle connector during the manufacturing process, shipping, or during the filling and injection steps that occur during use of the syringe.

The needle connector 26 has a channel 42 which extends from the proximal side 44 of the needle connector to its distal side 46. The channel 42 has an enlarged portion 48 adjacent to the proximal side 44, with two circumferential grooves 50, for engaging the plunger latching members, as described below.

The needle connector 26 and needle hub 28 attach securely together by means of circumferentially-disposed ribs 39 on the needle connector which interfit tightly with mating grooves 41 on the needle hub. Thus, when the needle connector is withdrawn into the barrel, the needle hub and needle are likewise retracted. The needle connector and the needle hub, being affixed together, form an assembly 40 having a cavity 52 therein. Part of the cavity comprises the channel 42 extending between the proximal and distal faces of the needle connector. Another part of the cavity 52 is the portion 53, within the needle connector, configured to hold the annular base portion 54 of the sealing ring 56. Another part of the cavity 52 is the portion 55 within the needle hub, between the distal end of the needle connector and the proximal end of the needle.

The sealing ring 56 has an elastically-deformable, inwardly- extending sealing portion 58, extending from the annular base portion 54, having an opening 60 therein. A movable sealing member 62 has a shaft portion 64 and a head portion 66. The shaft portion is posi- tioned in the channel 42 of the needle connector. The head 66 of the movable sealing member has a diameter larger than the diameter of the opening 60 of the sealing ring 56. The sealing portion 58 of the sealing ring is distally inwardly inclined and is elastically deformable such that the head 66 of the movable sealing member can pass through the opening 60 in the distal direction, and pass into the portion 55 of the cavity 52 in the needle hub under the pressure of liquid caused when the plunger in the syringe is moved in the distal direction.

The channel 42 in the needle connector 26 has four longitudinally-extending grooves 43 along the sides of the part of the channel 42 that accommodates the movable sealing member 62. The grooves permit the flow of liquid along the channel 42 past the sealing member 62. Thus, liquid can flow through the channel 42 for filling the syringe and for injection. In the cavity portion 55 in the needle hub, channels 57 are provided in the cavity walls, adjacent to the distal end of the cavity, to permit the flow of liquid past the head 66 of the sealing member when the head 66 is in the cavity portion 55.

Two locking pins 76 extend distally from the distal face 78 of the piston, proximate to its center, and aligned with the opening 70 of the channel 42 at the proximal side of the needle connector. The locking pins 76 are sized and positioned such that, when the distal face 78 of the piston is brought into contact with the proximal side 44 of the needle connector, the locking pins 76 are within the enlarged portion 48 of the channel 42 and the two ribs 80 on the radially outer surfaces of the locking pins are fitted in respective grooves 50 of the channel 42, in a snap-fit engagement therewith.

The barrel 22 is designed to prevent removal of the needle after use. A circumferential groove 82 and lip 84 are provided in the inner surface of the barrel wall 23 close to its proximal end, to engage the radially outer periphery 36 of the needle connector and the O-ring respectively, when the plunger is retracted after use. A frangible or weakened portion 86 of the plunger shaft 29 is provided to permit the shaft to be broken off by a user when the needle is retracted to the locked position within the barrel.

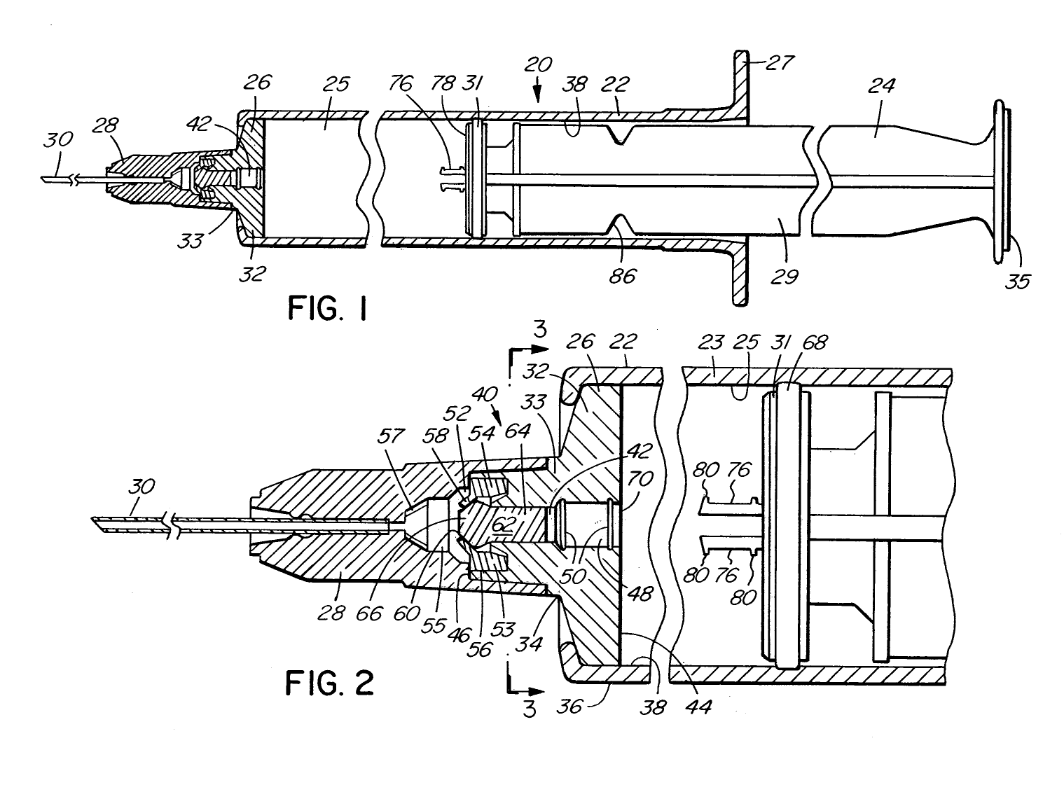

For initial filling of the syringe, the sealing member 62 is on the proximal side of the sealing ring 56, as shown in FIG. 2. Liquid can then be drawn into the barrel bore by movement of the plunger in the proximal direction, the liquid passing through the needle into the cavity portion 55, through the opening 60 in the sealing ring 56, through the channel 42 in the needle connector 26 and into the barrel bore. When the plunger is then pressed in the distal direction to inject the liquid, the liquid flows from the barrel bore through the channel 42, pressing against the proximal side of the sealing portion 58 of the ring 56 and against the proximal end of the movable sealing member 62. This pressure causes the opening 60 of the sealing ring 56 to enlarge and permit the head 66 of the movable sealing member 62 to be pushed through the opening and also permit the liquid to flow through the opening 60 in the distal direction, through the cavity portion 55 in the needle hub and out through the needle. This position is shown in FIG. 4.

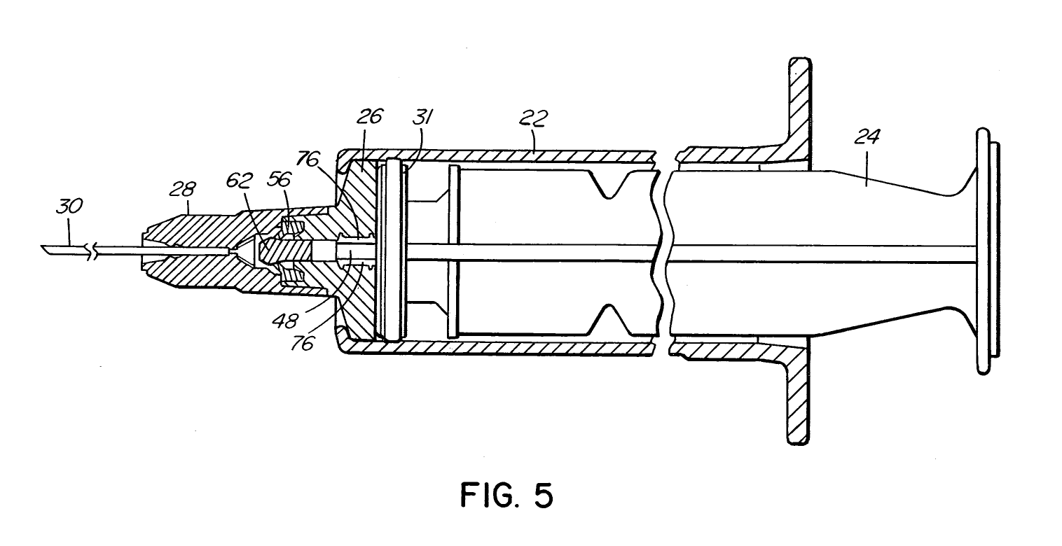

From this point, movement of the movable sealing member 62 in the proximal direction is stopped by the engagement of the sealing portion 58 of the ring 56 with the head 66 of the movable sealing member. The sealing portion 58, and more specifically its sealing surface 90 around the opening 60, seals against the head 66, stopping any flow of liquid in the proximal direction. The sealing member and sealing ring thus act as a one-way valve in the syringe, a mechanism which does not require that the plunger be in the fully inserted position in order to prevent refilling. As the plunger is pushed farther to complete the injection, emptying the barrel, the locking pins 76 enter the enlarged portion 48 of the channel 42 of the needle connector. The ribs 80 lock into the grooves 50 of the channel 42. The needle connector 26 is thus securely attached to the plunger 24, as illustrated in FIG. 5.

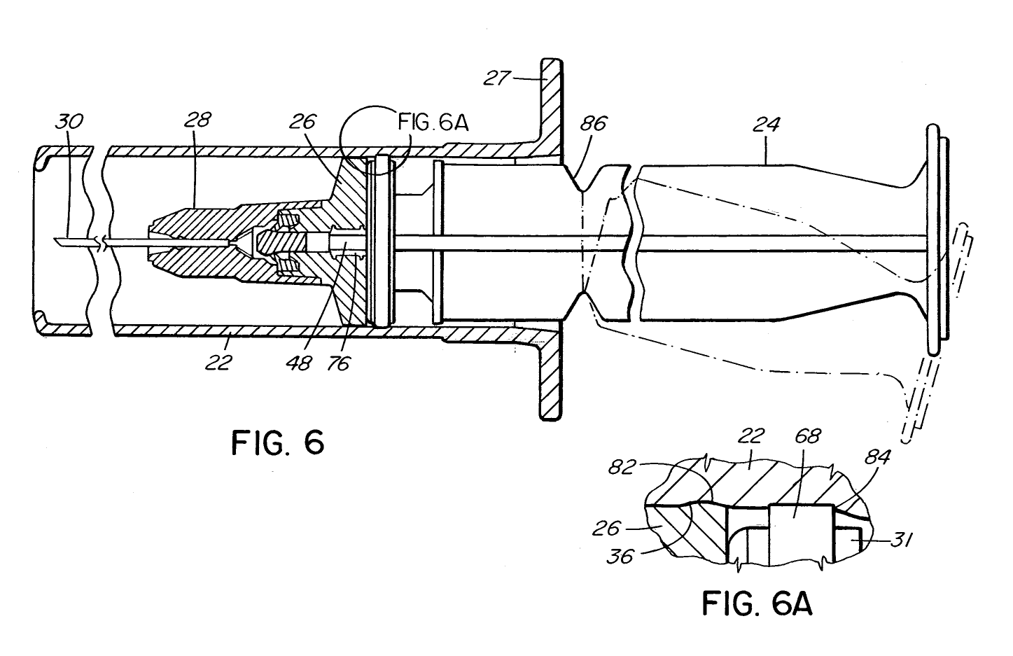

The plunger 24 is then retracted. As the plunger is moved in the proximal direction, the snap-fit attachment of the needle connector to the barrel wall 23 at the distal end of the barrel is overcome, permitting the needle connector to be withdrawn into the barrel bore, bringing with it the attached needle hub, and thus withdrawing the needle into the bore. This position is shown in FIG. 6. The plunger is withdrawn until the radially outer periphery 36 of the needle connector and the O-ring 68 engage the barrel circumferential groove 82 and circumferential lip 84 respectively, locking the plunger in place. The length of the needle is such that the needle is now in a fully retracted position within the barrel bore. The user then breaks off at the weakened point 86 the portion of the shaft extending beyond the proximal end of the barrel. The syringe may then be safely discarded.

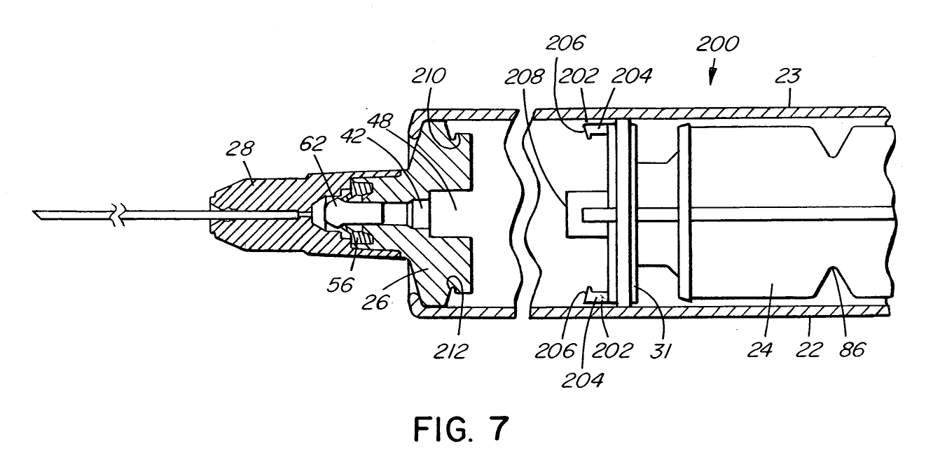

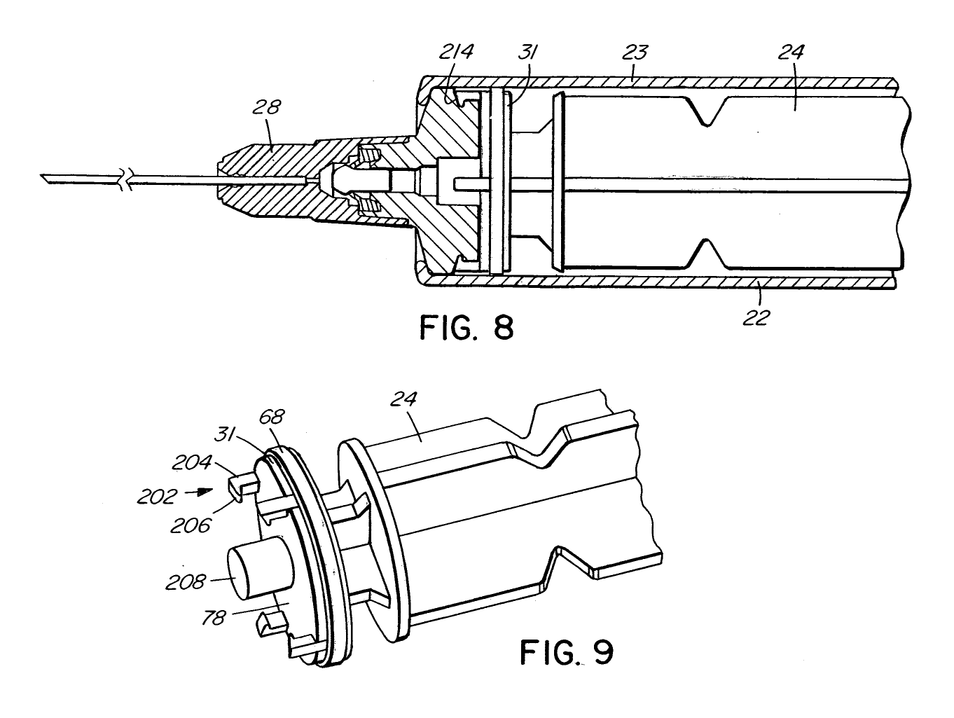

A second embodiment of the safety syringe is shown in FIGS. 7 to 9. The syringe 200 is essentially the same as the syringe 20 described above, except for the mechanism for locking together the needle connector and the plunger, and a structure for minimizing the volume of liquid left in the syringe after use.

The distal face 78 of the plunger has four locking pins 202 disposed at its periphery and extending distally and equally spaced apart. Each locking pin 202 has a leg 204 and an inwardly-extending hook portion 206. A projecting member, cylindrical post 208, extends distally from the center of the face 78. The needle connector 26 has four peripheral recesses 210, sized and spaced to be engaged by respective locking pins 202, with a lip 212 for engagement by the hook 206 of the pin 202. The channel 42 of the needle connector has an enlarged portion 48 sized to receive the post 208 of the plunger.

When the plunger is moved distally to engage the needle connector, the sloping surface 214 on the top of the resilient pins 202 causes the pins to be bent outwardly, into the space between the pins and the barrel wall 23. The pins resile inwardly as the hooks 206 fit around the lip 212 of the needle connector recesses. The post 208 of the plunger then occupies the volume of the channel enlargement 48, thus minimizing the volume of liquid left in the needle connector after use.

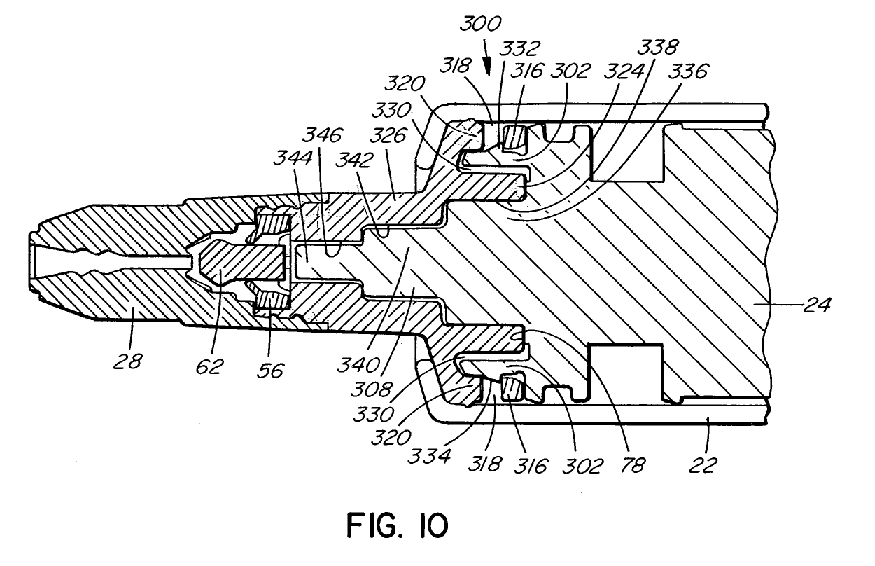

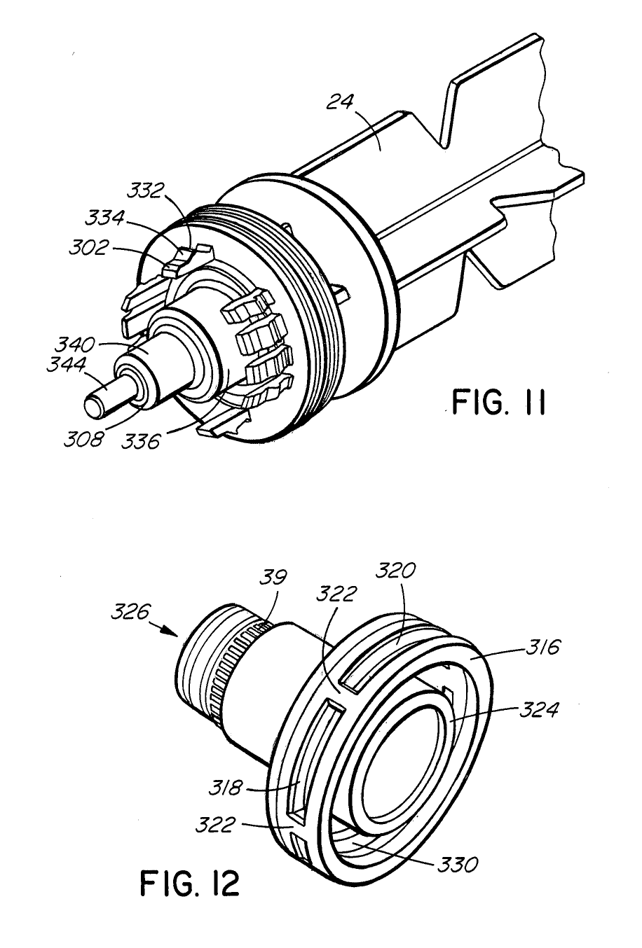

A third embodiment of the safety syringe is shown in FIGS. 10 to 12. The syringe 300 is essentially the same as the syringe 20 described above, except for the mechanism for locking together the needle connector and the plunger, and a structure for minimizing the volume of liquid left in the syringe after use.

The distal face 78 of the plunger has ten locking pins 302 disposed radially inwardly of its periphery and extending distally. The locking pins are arranged in four sets around a distally-extending post 308. Two opposed sets have four locking pins each. The other two opposed sets have a single locking pin each. Each locking pin 302 has an outwardly-projecting hook 332 subtended by a sloping surface 334 on the outward surface of the pin.

The needle connector 326 has an annular member 316 that is spaced by a radially-extending slot 318 from a flange 320. The annular member 316 is joined to the flange 320 by four connectors 322, dividing the slot 318 into four segments.

The needle connector 326 has a cylindrical wall 324 extending distally from its proximal end, the wall 324 defining a first enlarged portion 338 of the channel 42 of the needle connector. The wall 324 is radially spaced from the annular member 316 and the flange 320 by an axially-extending annual groove 330, which is sized to receive the locking pins 302.

The post 308 on the distal face of the piston has three sections which are successively smaller in the distal direction and are shaped to be received within respective sections of the channel 42 of the needle connector. The first section 336, being the largest in diameter, fits within the first section 338 of the channel; the second section 340 of the post 308 fits within the second section 342 of the channel; and the third section 344 of the post, being the smallest in diameter, fits within the smallest section 346 of the channel.

When the plunger is moved distally to engage the needle connector, the sloping outer surface 334 of the locking pins slides over the inner face of the annular member 316, causing the locking pins to bend inwardly. As the hooks fit into the slot 318 the pins resile outwardly so that the hooks engage the annular member 316. The spacing of the locking pins 302 and of the connectors 322 is such that, although some pins may align with a connector and not engage in the slot 318, at least four pins will engage in the slot, irrespective of the orientation of the plunger, forming a sufficiently secure attachment between the plunger and the needle connector for retraction of the needle.

In the locked position, the post 308 of the plunger occupies the volume of the three sections 338, 342, 346 of the channel 42, minimizing the volume of liquid left in the needle connector after use.

The components of the syringe may be made of any suitable materials. Such materials include polystyrene, polypropylene or polyethylene for the needle connector and the needle hub, acrylonitrile butadiene styrene (ABS) for the movable sealing member, thermoplastic elastomer (TPE) for the sealing ring, and polypropylene for the barrel and plunger.

As will be apparent to those skilled in the art in the light of the foregoing disclosure, many alterations and modifications are possible in the practice of this invention without departing from the scope thereof.

### LIST OF REFERENCE NUMERALS IN THE DRAWINGS

20 syringe
22 barrel
23 barrel wall
24 plunger
25 bore of barrel
26 needle connector
27 flange of plunger
28 needle hub
29 shaft of plunger
30 needle
31 piston
32 first portion of needle connector
33 second portion of needle connector
34 opening in distal end of barrel
35 thumb rest of plunger
36 outer periphery of needle connector
38 inner surface of barrel wall
39 ribs on needle connector
40 assembly of needle connector and needle hub
41 grooves on needle hub
42 channel in needle connector
43 longitudinal grooves along channel 42
44 proximal side of needle connector
46 distal side of needle connector
48 enlargement in channel 42
50 radial grooves in channel 42
52 cavity in assembly
53 cavity portion for sealing ring
54 annular base of sealing ring
55 cavity portion in needle hub
56 sealing ring
57 channels in cavity portion 55
58 sealing portion of sealing ring
60 opening in sealing portion of sealing ring
62 movable sealing member
64 shaft of movable sealing member
66 head of movable sealing member
68 O-ring
70 opening at distal end of channel 42
76 locking pins
78 distal face of plunger
80 ribs on locking pins
82 groove in barrel wall
84 lip in barrel wall
86 frangible portion of plunger shaft
90 sealing surface of sealing ring
200 syringe (second embodiment)
202 locking pins
204 leg of locking pin
206 hook of locking pin
208 post on distal face of piston
210 peripheral recesses of needle connector
212 lip of peripheral recess
214 sloping surface of locking pin
300 syringe (third embodiment)
302 locking pins
308 post on distal face of piston
316 annular member of needle connector
318 radial slot in needle connector
320 flange
322 connectors
324 cylindrical wall of needle connector
326 needle connector
330 annular groove in needle connector
332 hook of locking pin
334 sloping outer surface of locking pin
336 first section of post 308
338 first section of channel 42
340 second section of post
342 second section of channel
344 third section of post
346 third section of channel

1. A single use hypodermic syringe (20, 200, 300), comprising:
a barrel (22) having a barrel wall (23) defining a bore (25);
a plunger (24) slideably movable within the barrel;
a needle connector (26, 326) at the distal end of the barrel having a first portion (32) within the barrel bore and a second portion (33) extending from an opening (34) in the distal end of the barrel, the needle connector being in sealing, releasable engagement with the barrel wall;
a needle huh (28) affixed to the needle connector and having a hypodermic needle (30) fixed thereto;
the needle connector and the needle hub forming an assembly (40), the assembly having a cavity (52) therein, the needle and the barrel bore being in fluid communication through the cavity;
an elastically-deformable sealing ring (56) in the cavity having an opening (60) therein;
a sealing member (62) in the cavity having a head (66) and a shaft (64), the sealing member be movable in the cavity, by means of fluid movement in the cavity, from a first position in which the head is on the proximal side of the ring to a second position in which the head is on the distal side of the ring;
the ring being configured to stop movement of the sealing member from the second position to the first position and to form a seal with the sealing member, when the sealing member is in the second position, against a flow of fluid in the proximal direction;
locking means for locking together the plunger and the needle connector;
the needle being retractable into the barrel bore, when the plunger and the needle connector are locked together, by movement of the plunger in the proximal direction.

2. A syringe according to claim 1, wherein the locking means comprises at least one latching member (76) on the plunger at its distal face and at least one mating receptacle (42) for the latching member in the needle connector.

3. A syringe according to claim 1, wherein the locking means comprises at least two latching members (76) on the plunger at its distal face and positioned to fit in the cavity, and attachment means in the cavity to engage the latching members.

4. A syringe according to claim 3, wherein the attachment means comprises circumferential grooves (50) in the cavity wall, within the needle connector.

5. A syringe (200) according to claim 1, wherein the locking means comprises a plurality of latching members (202) on the plunger at its distal face, proximate to the radially outer periphery thereof, and mating attachment means (210, 212) for the latching members in the needle connector.

6. A syringe according to claim 5, further comprising a projecting member (208) on the distal face of the plunger adapted to be received in the cavity, within the needle connector.

7. A syringe according to claim 6, wherein the projecting member (208) displaces a volume of fluid in the cavity.

8. A syringe (300) according to claim 1, wherein the locking means comprises a plurality of latching members (302) on the plunger (24) at its distal face (78), spaced from the radially outer periphery thereof, and mating attachment means (316, 318, 330) for the latching members in the needle connector (326).

9. A syringe according to claim 8, wherein the latching members (302) have radially outwardly-extending projections (332).

10. A syringe according to claim 9, wherein the projections (332) are subtended by a sloping surface (334) on the outward surface of the latching members (302) for engagement with an annular member (316) of the needle connector.

11. A syringe according to claim 9, wherein the mating attachment means comprises an axially-extending groove (330) in the needle connector to receive the latching members (302) and a radially-extending slot (318) in the needle connector to receive the projections (332) on the latching members.

12. A syringe according to claim 11, wherein the radially-extending slot (318) in the needle connector (326) comprises a plurality of segments.

13. A syringe according to claim 11, wherein the mating engagement means in the needle connector (326) further comprises an annular member (316) spaced by the radially-extending slot (318) from a flange (320), the annular member (316) being joined to the flange (320) by connectors (322).

14. A syringe according to claim 13, wherein the connectors (322) divide the radially-extending slot (318) into segments.

15. A syringe according to claim 14, wherein there are tour connectors (322) and four segments of the radially-extending slot (318).

16. A syringe according to claim 13, wherein the projections (332) of the latching members (302) fit into the radially-extending slot (318) to engage the annular member (316) of the needle connector (326).

17. A syringe according to claim 16, wherein the projections (332) of at least four latching members (302) fit into the radially-extending slot (318) to engage the annular member (316) of the needle connector (326).

18. A syringe according to claim 13, wherein the needle connector (326) has a cylindrical wall (324) extending distally from its proximal end, the wall (324) being radially spaced from the annular member (316) and the flange (320) of the needle connector (326) by the axially-extending annular groove (330).

19. A syringe (300) according to claim 8, further comprising a projecting member (308) on the distal face of the plunger adapted to be received in the cavity, within the needle connector.

20. A syringe according to claim 19, wherein the projecting member (308) comprises three cylindrical sections (336, 340, 344) arranged axially with respect to each other and having different diameters.

21. A syringe according to claim 20, wherein the projecting member (308) displaces a volume of fluid in the cavity.

22. A syringe according to claim 1, wherein the needle connector (26) engages the barrel wall (23) with a snap-fit engagement between the radially outer periphery of the first portion (32) of the needle connector and the barrel wall.

23. A syringe according to claim 1, wherein the cavity has a first portion (42) thereof within the needle connector and a second portion (55) thereof within the needle hub, and in the second position of the sealing member (62) the head (66) is in the second portion of the cavity.

24. A syringe according to claim 1, wherein the needle connector has a cylindrical ring-retaining portion (53) at the distal end thereof.

25. A syringe according to claim 24, wherein the sealing ring (56) has an annular base portion (54) and an inwardly-extending sealing portion (58), the sealing ring opening (60) being in the inwardly-extending scaling portion thereof, the annular base portion being fitted into the ring retaining portion (53) of the needle connector.
